(19) United States
(12) Patent Application Publication
(10) Pub. No.: US 2026/0272887 A1
(43) Pub. Date: Sep. 17, 2026

(54) PHARMACEUTICAL COMPOSITION COMPRISING FLURALANER, MOXIDECTIN AND PRAZIQUANTEL FOR TREATING PARASITIC INFESTATIONS IN SMALL ANIMALS

(71) Applicant: AGROVET MRKET S.A., San Luis, Lima (PE)

(72) Inventors: Karen Melissa DELGADO CRISPIN, San Juan de Miraflores, Lima (PE); Erick Alfredo MOLLEAPAZA AGUILAR, Segunda etapa, Lima (PE); Mauricio Yvan PORRAS NICHO, San Juan de Lurigancho, Lima (PE); Jorge Umberto CALDERON OJEDA, La Molina Vieja, Lima (PE)

(73) Assignee: AGROVET MRKET S.A., San Luis, Lima (PE)

(21) Appl. No.: 19/156,843

(22) PCT Filed: Mar. 17, 2023
(86) PCT No.: PCT/PE2023/050003

§ 371 Date: Aug. 14, 2025

(30) Foreign Application Priority Data
Feb. 24, 2023 (CL) 000724-2023/DIN

(51) Int. Cl. A61K 31/42; A61K 9/20; A61K 31/198; A61K 31/365; A61K 31/4985; A61K 31/7076

(52) U.S. Cl. CPC A61K 31/42; A61K 9/2013; A61K 9/2031; A61K 9/2054; A61K 9/2059; A61K 9/2095; A61K 31/198; A61K 31/365; A61K 31/4985; A61K 31/7076

(57) ABSTRACT

The present invention discloses a pharmaceutical formulation or composition and a method for manufacturing or producing same, wherein the pharmaceutical composition comprises fluralaner, moxidectin and praziquantel for treating parasitic infestations in animals. The composition may be in tablet form, more particularly as a chewable tablet, and more preferably as a soft chewable tablet that masks the bitter flavour of the active ingredient praziquantel based on a series of bitter taste receptor blockers.

### TECHNICAL FIELD

The present invention pertains to the technical field of the pharmaceutical industry, mainly with the industry of pharmaceutical products for veterinary use.

### PRIOR ART

The development of pharmaceutical compositions or formulations comprising fluralaner, moxidectin and praziquantel is a current need for the treatment and control of infestations in small animals, for the treatment of parasitosis caused by flatworms and other species of roundworms.

The document WO2022/051478 is known to disclose oral dosage formulations comprising an effective amount of an isoxazoline parasiticidal agent, an avermectin and a pyrazinoisoquinoline, and optionally one or more additional active ingredients, such as a tetrahydropyrimidine. The formulation may comprise fluralaner, praziquantel and moxidectin together with microcrystalline cellulose (25% to 30%), lactose monohydrate (5% to 6%), butyl hydroxytoluene (0.05-0.06%), croscarmellose sodium (3 to 5%), sodium lauryl sulfate (0.4 to 0.5%), flavoring (5 to 20%), magnesium stearate (0.5 to 1.5%) and colloidal silicon dioxide) in palatable dosage forms for veterinary use wherein the composition has a first coated granule comprising praziquantel and moxidectin together with a diluent, an antioxidant and a disintegrant in a physiologically acceptable polymeric matrix and a second granule comprising an isoxazoline, primarily lotilaner together with a diluent, a wetting agent, a disintegrant and a binder.

Also known is document WO2021/046305 disclosing a palatable granular veterinary composition comprising at least one active agent, at least one wetting agent and at least one flavoring agent for controlling or treating a condition in an animal, wherein the composition may comprise praziquantel and moxidectin at a concentration from 0.001% to 75% based on the total weight of the palatable granular composition and may contain glycerin, fatty acids, polyethylene glycol, croscarmellose sodium and microcrystalline cellulose as disintegrants, sodium lauryl sulfate and magnesium stearate as lubricants, hydroxypropyl methylcellulose as binders, butylated hydroxy anisole and butylated hydroxy toluene as antioxidants and parabens as buffering agents.

Also known is document WO2021/046296, which provides a palatable veterinary soft chewing composition comprising at least one active agent, at least one wetting agent and at least one flavoring, wherein the composition may comprise praziquantel and moxidectin and may further contain glycerin, fatty acids, polyethylene glycol, croscarmellose sodium and microcrystalline cellulose as disintegrants, sodium lauryl sulfate and magnesium stearate as lubricants, hydroxypropyl methylcellulose as a binding agent (binder), butyl hydroxy anisole and butyl hydroxy toluene as antioxidants and parabens as buffering agents.

Patent WO2020/051106 discloses a palatable hard chewable composition comprising at least one veterinarily acceptable isoxazoline, a macrocyclic lactone, an acceptable salt form of pyrantel, at least one flavor of natural animal origin and at least one veterinarily acceptable excipient. The composition is compressed into a hard tablet. Isoxazoline can be sarolaner, afoxalaner, fluralaner or lotilaner, stabilized moxidectin (macrocyclic lactone stabilized with an antioxidant) and an antiparasitic such as praziquantel. The composition may have as excipients croscarmellose sodium (disintegrant), polyethylene glycol, sodium lauryl sulfate, hydroxypropyl methylcellulose as stabilizer, butyl hydroxyanisole and butyl hydroxy toluene as antioxidants, magnesium stearate as lubricant.

Patent EP2662075 relates to improvements in oral formulations of the anthelmintic praziquantel used for the control and treatment of endoparasite infestations in warm-blooded animals in particular in companion animals such as cats, dogs and horses wherein the composition comprises moxidectin-containing praziquantel particles with an integral coating of a lipid or a mixture of lipids which are insoluble in water and which serve to mask the bitter taste of praziquantel. The composition may comprise starch and purified water.

In this sense, there is still a need to provide a pharmaceutical composition or formulation comprising an association of active ingredients that can provide comprehensive protection against external parasites, such as fleas, ticks, and mites, as well as internal parasites, including Dirofilariasis, gastrointestinal nematodes, and tapeworms, among others. The proposed composition contains an active of the isoxazoline family, an avermectin and a synthetic isoquinoline-pyrazine derivative.

The proposed composition is a broad-spectrum parasiticide containing a combination of fluralaner, moxidectin, and praziquantel, available for oral administration. The composition for oral administration, where the masking of the bitter taste of one of the active ingredients is achieved, in the form of a tablet, more particularly in a soft chewable tablet having a high palatability.

### BRIEF DESCRIPTION OF THE INVENTION

The present invention is directed to a pharmaceutical formulation or composition and a method of manufacture or production thereof, wherein the pharmaceutical composition comprises fluralaner, moxidectin and praziquantel for the treatment of parasitic infestations in animals, wherein the composition may be in tablet form, more particularly the latter as a chewable tablet and more preferably as a soft chewable tablet which manages to mask the bitter taste of the active principle praziquantel on the basis of an association of bitter taste receptor blockers.

### DETAILED DESCRIPTION OF THE INVENTION

In a first aspect the present invention refers to a pharmaceutical composition or formulation of fluralaner associated with moxidectin and praziquantel for the treatment of parasitic infestations in minor animals, wherein the composition comprises fluralaner, praziquantel, moxidectin, together with pharmaceutically acceptable excipients for oral administration.

The present invention refers to a palatable oral pharmaceutical composition or formulation of fluralaner associated with moxidectin and praziquantel for the treatment of parasitic infestations in minor animals, wherein the composition comprises fluralaner, praziquantel, moxidectin, together with pharmaceutically acceptable excipients such as flavor enhancers, diluents, disintegrants, humectants, plasticizers, solvents, surfactants, flavor masking agents, sweeteners, binders, lubricants, preservatives, antioxidants, and mixtures thereof.

In this sense, the active ingredients may be present in a concentration between 0.1% and 40% based on the total weight of the composition. In one embodiment of the invention, the active principle fluralaner may be in a concentration between 5% and 30% in relation to the total weight of the final pharmaceutical composition, for oral administration it may preferably be between 10% and 15% in relation to the total weight of the final pharmaceutical composition. The active ingredient praziquantel may be present in the composition at a concentration between 0.1% to 10% relative to the total weight of the final pharmaceutical composition, for oral administration it may preferably be between 1% to 5% relative to the total weight of the final pharmaceutical composition. The active ingredient moxidectin is at a concentration of 0.10% to 5% relative to the total weight of the final pharmaceutical composition, for oral administration it may preferably be at a concentration of 0.10 to 1% relative to the total weight of the final pharmaceutical composition.

In relation to the pharmaceutically acceptable excipients or components which may be in the pharmaceutical composition or formulation of the present invention said excipients are selected from the group consisting of flavor enhancers, diluents, disintegrants, humectants, plasticizers, solvents, surfactants, flavor masking agents, sweeteners, binders, lubricants, preservatives, antioxidants, and mixtures thereof.

As for the flavor enhancer, it can be a flavoring or a mixture of them, which include flavorings of natural animal origin, which can be pork, chicken or beef and/or added to flavorings of synthetic origin that improve odor, these can also be of meat flavor. The flavor enhancer(s) may be present in the pharmaceutical composition or formulation of the present invention in a proportion between 5% and 30% relative to the total weight of the final pharmaceutical composition.

In addition to the diluent or disintegrant, the pharmaceutical composition according to the present invention may further comprise a solvent, a preservative, antioxidants and a cosolvent or diluent.

The diluent or disintegrant which may be in the pharmaceutical composition according to the present invention, may be sodium starch glycolate, sodium alginate, sodium alginate, calcium alginate, microcrystalline cellulose, pregelatinized corn starch, corn starch, which can be in a range between 5% and 40% in relation to the total weight of the final composition. In a preferred embodiment of the invention, the disintegrant may comprise in addition to starch, croscarmellose sodium in a proportion between 0.5% and 5% in relation to the total weight of the final pharmaceutical composition.

The composition according to the present invention may further comprise humectants, such as oils of vegetable origin such as soybean oil, sunflower oil, canola oil and mixtures thereof. Oils as humectants can be in a proportion between 5% and 25% in relation to the total weight of the final composition. In one embodiment of the invention, the composition may additionally contain as a humectant, glycerin in a proportion between 5% and 20% in relation to the total weight of the final composition.

The pharmaceutical composition in accordance with the present invention comprises a forming agent or plasticizer to provide the final tablet texture polyethylene glycol of a suitable molecular weight, which preferably should be equal to or greater than soft, such as polyvinylpyrrolidone, hydroxypropylmethylcellulose and polyethylene glycol, preferably 1000 g/mol in the present pharmaceutical composition the plasticizer may be in a concentration between 5% and 15% on a weight basis of the final pharmaceutical composition.

The pharmaceutical composition according to the present invention may further contain other solvents and diluents. In a preferred embodiment of the invention, the additional diluent may be microcrystalline cellulose in a concentration between 2% and 10% relative to the total weight of the final composition and more preferably 2.5% by weight based on the total weight of the final pharmaceutical composition. The additional solvent may be propylene glycol at a concentration between 1% and 10% relative to the total weight of the final composition, more preferably 2.5% by weight based on the total weight of the final pharmaceutical composition.

The pharmaceutical composition in accordance with the present invention may further comprise a surfactant, preferably an anionic surfactant which may be dodecylbenzene sulfonate, sodium dioctylsulfosuccinate or sodium lauryl sulfate in a concentration between 0.5% and 3.0% relative to the total weight of the final composition, more preferably 2.0% based on the total weight of the final pharmaceutical composition. In a preferred embodiment of the present invention, the surfactant is sodium lauryl sulfate.

The pharmaceutical composition according to the present invention may further comprise one or more bitter taste blockers. Bitter taste blockers can be adenosine 5′-monophosphate (AMP), thymidine 5′-monophosphate (TMP), adenosine 5′-triphosphate (ATP), adenosine 5′-triphosphate (ATP), thymidine 5′-monophosphate (TMP) and adenosine 5′-monophosphate (ATP), monosodium glutamate and mixtures thereof, in a concentration between 0.10% and 1.0%, more preferably 0.50%, based on the total weight of the final pharmaceutical composition.

The pharmaceutical composition according to the present invention may further comprise one or more sweeteners, such as saccharin, sorbitol, aspartame, and sucralose in a concentration between 0.1% and 2% based on the total weight of the final pharmaceutical composition.

The pharmaceutical composition according to the present invention may further comprise one or more binders such as copovidone, crospovidone, povidone, carboxymethylcellulose, hydroxypropylmethylcellulose in a concentration between 0.1% and 1% based on the total weight of the final pharmaceutical composition.

The pharmaceutical composition in accordance with the present invention may further contain one or more lubricants, such as magnesium stearate, in a concentration between 0.1% and 1.0%, more preferably 0.50% based on the total weight of the final pharmaceutical composition.

The pharmaceutical composition according to the present invention may further contain one or more preservatives such as sodium sulfite, sodium metabisulfite, ethyl paraben, methyl paraben and propyl paraben, in a concentration between 0.01% and 0.10% relative to the total weight of the final composition. In a preferred embodiment of the invention, the preservatives are methyl paraben at a concentration of 0.05% and propyl paraben at a concentration of 0.02% based on the total weight of the final pharmaceutical composition.

The pharmaceutical composition in accordance with the present invention may further contain antioxidants, which may be one or more synthetically produced antioxidants, such as propylgallate (PG), tertiary butylhydroxyquinone (TBHQ), hydroxybutylanisole (BHA) and butylated hydroxytoluene (BHT) and may be in the pharmaceutical composition or formulation in a proportion between 0.005 and 0.05% by weight of the final formulation and more preferably at 0.04% relative to the total weight of the final composition.

The pharmaceutical composition according to the present invention may further contain purified water as a solvent during the process of obtaining the formulation in a concentration between 1.0% and 10% plus preferably a concentration of 7.0% based on the weight of the composition prior to its obtaining, wherein the water evaporates during the process. Also, the composition may comprise as an additional solvent ethyl alcohol (ethanol) in a concentration of 1.50% based on the weight of the composition before it is obtained which is evaporated during the process.

The pharmaceutical composition in accordance with the present invention is for veterinary use and may be in a pharmaceutically acceptable form for oral administration such as a tablet, more preferably as a chewable tablet and more preferably as a highly palatable soft chewable tablet wherein the unpleasant taste of the active principle praziquantel is masked by the presence of bitter taste receptor blockers, wherein said blockers are adenosine 5′-monophosphate and monosodium glutamate.

In a second aspect, the present invention refers to a method of producing a pharmaceutical composition comprising fluralaner, moxidectin and praziquantel for the treatment of parasitic infestations in animals, wherein the method comprises the following steps:
a) Dissolving the binder(s) with ethyl alcohol;
b) Dissolving the sweetener in purified water;
c) Mixing the product of step 2 with the product of step 1;
d) Mixing praziquantel with the diluent (microcrystalline cellulose) and the bitter taste blockers;
e) Performing wet granulation with the result of steps c) and d) and dry at 45 degrees Celsius;
f) Dissolving Moxidectin in propylene glycol as solvent;
g) Adding to step f) the preservatives and continue with the dissolution;
h) Dispersing the flavor enhancer(s) in glycerin for a period of 2 minutes to 15 minutes
i) Dissolving the antioxidants in the moisturizer (soybean oil);
j) Mixing the result of step e) with fluralaner, flavor improvers, diluent/disintegrant (starch), disintegrant (croscarmellose sodium), surfactant, flavor blockers, sweetener, and lubricant;
k) Adding the product of step h) to the product of step i) and kneading;
l) Adding on the result of step j), the result of step g) and glycerin;
m) Adding to the result of step k) polyethylene glycol; kneading and
n) Carrying out the tabletting.

In step c), mixing is carried out until total homogenization during a period of 60 minutes, preferably 30 minutes.

In step d) mixing with the other components for a period of 30 minutes, more preferably 15 minutes.

In step e) the drying time is between 6 hours and 10 hours, preferably 8 hours.

In step f) the dissolution is carried out in a suitable vessel until complete dissolution for a period of 1 minute to 30 minutes, more preferably 5 minutes.

In step g) the preservatives are added and the stirring is continued until complete dissolution, during a period of 20 minutes to 60 minutes, more preferably 25 minutes.

In step h) the dispersion of the flavor enhancer(s) is performed in a period of 2 to 15 minutes, more preferably 10 minutes.

In step i) the mixing of the antioxidants in the wetting agent is carried out in a suitable vessel and for a period of 30 minutes to 120 minutes, more preferably 60 minutes.

In step j) it is mixed for 5 minutes and placed in a kneading machine. Mixing is then continued for a period of 20 minutes to 120 minutes, more preferably 40 minutes.

In step k) the mixture is kneaded for a period of 30 to 60 minutes, preferably 45 minutes until the solid part is completely wetted.

In step l) the stirring is continued for an additional time period of 30 minutes to 60 minutes, more preferably 45 minutes until complete wetting/formation of a moldable mass.

In step m), in a suitable vessel, the excipient (propylene glycol) is heated until completely melted with constant stirring at a temperature between 60° C. and 70° C. and kneaded until a moldable dough is formed for a period of 30 minutes to 60 minutes, more preferably 45 minutes.

### EXAMPLES OF FORMULATION OR COMPOSITIONS ACCORDING TO THE INVENTION

Examples of the declared composition are shown below in Table N° 1. The water and alcohol solvents used in granulation evaporate during the process and are therefore not considered in the weight of the final composition.

TABLE NO 1
Examples of formulas with disclosed composition
Formulas Evaluated (%)

| Component | A | B | C | D | E | F |
|---|---|---|---|---|---|---|
| Fluralaner | 12.50% | 12.50% | 12.50% | 12.50% | 12.50% | 12.50% |
| Moxidectin | 0.10% | 0.10% | 0.10% | 0.10% | 0.10% | 0.10% |
| Praziquantel | 2.50% | 2.50% | 2.50% | 2.50% | 2.50% | 2.50% |
| Flavoring | 21.00% | 21.00% | 21.00% | 21.00% | 21.00% | 21.00% |
| Corn starch | 18.49% | 17.99% | 17.89% | 18.49% | 11.49% | 12.49% |
| Soybean oil | 12.00% | 12.00% | 12.00% | 12.00% | 12.00% | 11.00% |
| Glycerin | 10.00% | 10.00% | 10.00% | 10.00% | 10.00% | 10.00% |
| Polyethylene glycol 3350 | 10.00% | 10.00% | 10.00% | 10.00% | 10.00% | 10.00% |
| Croscarmellose sodium | 3.00% | 3.00% | 3.00% | 3.00% | 3.00% | 3.00% |
| Microcrystalline cellulose | 2.50% | 2.50% | 2.50% | 2.50% | 7.50% | 7.50% |
| Propylene glycol | 2.50% | 2.50% | 2.50% | 2.50% | 2.50% | 2.50% |
| Sodium lauryl sulfate | 2.00% | 2.00% | 2.00% | 2.00% | 2.00% | 2.00% |
| Sucralose | 1.00% | 1.00% | 1.00% | 1.00% | 1.00% | 1.00% |
| Adenosine 5′ | 0.50% | — | 1.00% | 1.00% | 1.00% | 1.00% |
| monophosphate | | | | | | |
| Adenosine disodium | 0.50% | 1.00% | — | 1.00% | 1.00% | 1.00% |
| triphosphate | | | | | | |
| Monosodium glutamate | 0.50% | 1.00% | 1.00% | 0.50% | 1.00% | 1.00% |
| Magnesium stearate | 0.50% | 0.50% | 0.50% | 0.50% | 0.50% | 0.50% |
| Hydroxypropyl | 0.30% | 0.30% | 0.40% | 0.30% | 0.80% | 0.80% |
| methylcellulose | | | | | | |
| Methyl paraben | 0.05% | 0.05% | 0.05% | 0.05% | 0.05% | 0.05% |
| Propyl paraben | 0.02% | 0.02% | 0.02% | 0.02% | 0.02% | 0.02% |
| Butyl hydroxy toluene | 0.02% | 0.02% | 0.02% | 0.02% | 0.02% | 0.02% |
| Butyl hydroxy anisole | 0.02% | 0.02% | 0.02% | 0.02% | 0.02% | 0.02% |
| Ethyl alcohol | 2.00% | 2.00% | 1.50% | 2.00% | 3.00% | 3.00% |
| Purified water | 3.00% | 3.00% | 2.00% | 3.00% | 4.00% | 4.00% |

* Evaporates during the manufacturing process

### Study Design

The palatability of the different formulas under evaluation was determined with 113 canines of both sexes, older than 8 weeks of age, of different weights, of different breeds and clinically healthy. The tablets were presented to the animals and a score was determined for each composition according to the reaction based on the scale shown in Table N° 2.

TABLE N°2
Acceptance scale in canines

| Score | Description |
|---|---|
| 0 | The animal must be forced to eat the tablet |
| 1 | The animal shows interest at first, but must be |
| | forced to eat the tablet |
| 2 | The animal shows interest and eats the tablet when |
| | it is placed on its snout |
| 3 | The animal smells the tablet and eats it voluntarily |

### Results

Table N° 3 shows the averages for each formula under evaluation, as well as the number of canines in which they were tested. With the exception of formula D, all formulas had an average score above 2.6, making them highly palatable. Formula C had an average score of 3, which places it as the most palatable formula of those evaluated. Overall, most of the tablets were well accepted. However, D had an average acceptance (74.19%), in contrast to the others, which had an acceptance of over 80%. C had a 100% acceptance rate. When a kruskal wallis test was performed to determine if there was a statistical difference between the groups, it was determined that there were only significant statistical differences between C and D.

TABLE NO 3
Acceptance results of the evaluated formulas

| | Accept | | Does not accept | | | No. of |
|---|---|---|---|---|---|---|
| | No. of | | No. of | | | animals |
| Formula | animals | % | animals | % | Score | treated |
| A | 27 | 96.4 | 1 | 3.6 | 2.82 | 28 |
| B | 26 | 86.7 | 4 | 13.3 | 2.63 | 30 |
| C | 24 | 100.0 | 0 | 0.0 | 3.00 | 24 |
| D | 23 | 74.2 | 8 | 25.8 | 2.23 | 31 |
| E | 24 | 86.1 | 4 | 14.0 | 2.67 | 28 |
| F | 26 | 92.9 | 2 | 7.1 | 2.79 | 28 |

According to the evaluation, it is evident that the combination of adenosine 5′-monophosphate with monosodium glutamate at a concentration of 2.0% is effective in blocking the bitter taste of the active ingredient praziquantel.

1-9. (canceled)

10. A pharmaceutical composition for treatment of parasitic infestations in small animals, characterized in that it comprises as active ingredients fluralaner, moxidectin and praziquantel, bitter taste receptor blockers and pharmaceutically acceptable excipients.

11. The pharmaceutical composition for treatment of parasitic infestations in minor animals according to claim 10, wherein the pharmaceutically acceptable excipients are selected from the group consisting of flavor enhancers, diluents, disintegrants, humectants, plasticizers, solvents, surfactants, flavor masking agents, sweeteners, binders, lubricants, preservatives antioxidants and mixtures thereof.

12. The pharmaceutical composition for treatment of parasitic infestations in minor animals according to claim 10, wherein the active ingredients are in a concentration between 0.1% and 40% based on the total weight of the composition.

13. The pharmaceutical composition for treatment of parasitic infestations in minor animals according to claim 10, wherein the bitter taste receptor blockers are selected from the group consisting of adenosine 5′-monophosphate (AMP), thymidine 5′-monophosphate (TMP), adenosine 5′-triphosphate (ATP), monosodium glutamate and mixtures thereof in a concentration between 0.10% and 1.0%.

14. The pharmaceutical composition for treatment of parasitic infestations in small animals according to claim 10, further comprising purified water as a solvent in a concentration between 1.0% and 10% based on the weight of the composition before it is obtained.

15. The pharmaceutical composition for treatment of parasitic infestations in small animals according to claim 10, further comprising as an additional solvent ethyl alcohol (ethanol) at a concentration of 1.50% based on the weight of the composition before it is obtained.

16. The pharmaceutical composition for treatment of parasitic infestations in minor animals according to claim 10, wherein the pharmaceutical composition is in the form of a tablet.

17. The pharmaceutical composition for treatment of parasitic infestations in minor animals according to claim 16, wherein the tablet is a soft chewable tablet.

18. A method of producing a pharmaceutical composition comprising fluralaner, moxidectin and praziquantel for the treatment of parasitic infestations in animals, wherein the method comprises:
a) Dissolving the binder(s) with ethyl alcohol;
b) Dissolving the sweetener in purified water;
c) Mixing the product of step b) with the product of step a);
d) Mixing praziquantel with the diluent (microcrystalline cellulose) and the bitter taste blockers;
e) Performing wet granulation with the result of steps c) and d) and dry at 45 degrees Celsius;
f) Dissolving Moxidectin in propylene glycol as solvent;
g) Adding to step f) the preservatives and continue with the dissolution;
h) Dispersing the flavor enhancer(s) in glycerin for a period of 2 minutes to 15 minutes;
i) Dissolving the antioxidants in the moisturizer (soybean oil);
j) Mixing the result of step e) with fluralaner, flavor improvers, diluent/disintegrant (starch), disintegrant (croscarmellose sodium), surfactant, flavor blockers, sweetener, and lubricant;
k) Adding the product of step h) to the product of step i) and kneading;
l) Adding on the result of step j), the result of step g) and glycerin;
m) Adding to the product of step k) polyethylene glycol; knead and
n) Carrying out the tableting.
